(19) United States
(12) Patent Application Publication
(10) Pub. No.: US 2014/0243943 A1
(43) Pub. Date: Aug. 28, 2014

(54) SYSTEM AND METHOD FOR STIMULATING INTRAOSSEOUS NERVE FIBERS

(71) Applicant: BOSTON SCIENTIFIC NEUROMODULATION CORPORATION, Valencia, CA (US)

(72) Inventors: Prakash Rao, Philadelphia, PA (US); Kerry Bradley, Glendale, CA (US)

(73) Assignee: BOSTON SCIENTIFIC NEUROMODULATION CORPORATION, Valencia, CA (US)

(21) Appl. No.: 14/188,267

(22) Filed: Feb. 24, 2014

Related U.S. Application Data
(60) Provisional application No. 61/768,935, filed on Feb. 25, 2013.

(51) Int. Cl. A61N 1/05

(52) U.S. Cl. CPC A61N 1/0551
USPC 607/116

(57) ABSTRACT

A method for treating a patient having pain comprises applying electrical modulation energy to a target site adjacent an intraosseous nerve fiber of the patient to modulate pain traffic within the intraosseous nerve fiber, thereby treating the pain.

### RELATED APPLICATION DATA

The present application claims the benefit under 35 U.S.C. §119 to U.S. provisional patent application Ser. No. 61/768,935, filed Feb. 25, 2013. The foregoing application is hereby incorporated by reference into the present application in its entirety.

### FIELD OF INVENTION

The present invention generally relates to electrical stimulation systems and methods, and more particularly, to an electrical stimulation system and method for treating chronic back pain.

### BACKGROUND OF THE INVENTION

Implantable neurostimulation systems have proven therapeutic in a wide variety of diseases and disorders. For example, Spinal Cord Stimulation (SCS) techniques, which directly stimulate the spinal cord tissue of the patient, have long been accepted as a therapeutic modality for the treatment of chronic neuropathic pain syndromes, and the application of SCS has expanded to include additional applications, such as angina pectoralis, peripheral vascular disease, and incontinence, among others. SCS may also be a promising option for patients suffering from motor disorders, such as spasticity, and neural degenerative diseases such as multiple sclerosis.

An implantable SCS system typically includes one or more electrode-carrying stimulation leads, which are implanted at a stimulation site in proximity to the spinal cord tissue of the patient, and a neurostimulator implanted remotely from the stimulation site, but coupled either directly to the stimulation lead(s) or indirectly to the stimulation lead(s) via a lead extension. The neurostimulation system may further include a handheld patient programmer to remotely instruct the neurostimulator to generate electrical stimulation pulses in accordance with selected stimulation parameters. The handheld programmer may, itself, be programmed by a technician attending the patient, for example, by using a Clinician's Programmer (CP), which typically includes a general purpose computer, such as a laptop, with a programming software package installed thereon.

Thus, programmed electrical pulses can be delivered from the neurostimulator to the stimulation lead(s) to stimulate or activate a volume of neural tissue. In particular, electrical stimulation energy conveyed to the electrodes creates an electrical field, which, when strong enough, depolarizes (or “stimulates”) the neural fibers within the spinal cord beyond a threshold level. This induces the firing of action potentials (APs) that propagate along the neural fibers to provide the desired efficacious therapy to the patient.

As discussed, SCS may be utilized to treat patients suffering from chronic neuropathic pain. To this end, electrical stimulation is generally applied to the dorsal column (DC) nerve fibers, which is believed to inhibit the perception of pain signals via the gate control theory of pain by creating interneuronal activity within the dorsal horn that inhibits pain signals traveling from the dorsal root (DR) neural fibers that innervate the pain region of the patient up through the spinothalamic tract of the spinal cord to the brain. Consequently, stimulation leads are typically implanted within the dorsal epidural space to provide stimulation to the DC nerve fibers. Thus, SCS has secured a place in the arsenal of many physicians, because of the analgesic effects it provides to patients with chronic pain. While many chronic pain patients benefit from SCS therapy, there are some who do not because of different pathophysiology and supraspinal processing.

Back pain is a multifactorial ailment affecting millions of people, requiring considerable expenditure of medical resources as well as imposing significant burden on those who suffer from this condition. Back pain may occur due to a wide variety of factors, and this condition can be highly refractive to treatment. It has been recognized that basivertebral nerves play a key role in chronic back pain. Basivertebral nerves are intraosseous nerves that enter the vertebral bodies through the posterior vascular foramen (“basivertebral foramen”), which is present at the posterior midline of all human thoracic and lumbar vertebrae, and innervates the trabecular bone of each vertebral body to supply vasomotor nerve signals to the blood vessels within each vertebral body.

In addition to vasomotor involvement, it has been found that the basivertebral nerves in the vertebrae may be capable of transmitting nociceptive traffic to the brain via spinal nerves. In particular, there is documented evidence that a peptide neurotransmitter (“substance P”), which is released in response to nociceptive stimuli, is present within the basivertebral nerves (see Fras C, Kravetz P., Mody D R, Heggeness M H, Substance P-Containing Nerves within the Human Vertebral Body, an Immunohistochemical Study of the Basivertebral Nerve. Spine J 2003; 3(1): 63-6). The basivertebral nerves are subjected to stress as a patient moves. Eventually, accumulated stress on the vertebrae can put pressure against these exposed nerves, causing severe back pain even during normal, everyday movement. The pain triggered by these nerves forces sufferers to avoid a variety of activities, taking a substantial toll on overall quality of life.

A number of treatment approaches have focused on the basivertebral nerves. Primarily, treatment approaches have focused on pharmacological solutions, providing a number of compounds aimed at stimulating the nociceptive traffic of the basivertebral nerves. A recent therapeutic development has suggested ablating some or all of the basivertebral nerve tissue in the affected area. However, this process is irreversible and carries the possibility of undesirable side effects.

Thus, a need remains for a process that can ameliorate back pain without permanently affecting the basivertebral nerves.

### SUMMARY OF THE INVENTION

In accordance with the present inventions, a method for treating a patient having pain is provided. The method comprises applying electrical modulation energy to a target site (e.g., a bone, such as vertebral body, pelvis, femur, fibula, humerus, ulna, radius, etc., in which the intraosseous nerve fiber innervates) adjacent an intraosseous nerve fiber of the patient to modulate pain traffic (e.g., nociceptive pain traffic) within the intraosseous nerve fiber, thereby treating the pain. In one method, intraosseous nerve fiber is a basivertebral nerve fiber, and the pain is back pain.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred exemplary embodiments of the present disclosure, in which similar elements are referred to by common reference numerals. In order to better appreciate how the above-recited and other advantages and objects of the present disclosure are obtained, a more particular description of the present disclosure briefly described above will be rendered by reference to specific exemplary embodiments thereof, which are illustrated in the accompanying drawings. Understanding that these drawings depict only typical exemplary embodiments of the disclosure and are not therefore to be considered limiting of its scope, the disclosure will be described and explained with additional specificity and detail through the use of the accompanying drawings in which:

FIG. 1 is a plan view of a neuromodulation system constructed in accordance with one exemplary embodiment of the present invention;

FIG. 2 is a plan view of the neuromodulation system of FIG. 1 in use within a patient;

FIG. 3 is a plan view of an implantable pulse generator (IPG) and three percutaneous modulation leads used in the neuromodulation system of FIG. 1;

FIG. 4A is a cross-sectional top view of a vertebra, wherein one of the modulation leads is used to directly modulate basivertebral nerve fibers within the vertebra in accordance with one exemplary technique of the present invention;

FIG. 4B is a cross-sectional top view of a vertebra, wherein one of the modulation leads is used to indirectly modulate basivertebral nerve fibers within the vertebra in accordance with one another exemplary technique of the present invention;

FIG. 5A is a top view of a vertebra illustrating a transpedicular approach used to deliver a neuromodulation lead into a body of the vertebra in proximity to the basivertebral nerve fibers;

FIG. 5B is a side view of a vertebra illustrating the transpedicular approach of FIG. 5A;

FIG. 6A is a top view of a vertebra illustrating a postereolateral approach used to deliver a neuromodulation lead into a body of the vertebra in proximity to the basivertebral nerve fibers; and

FIG. 6B is a side view of a vertebra illustrating the transpedicular approach of FIG. 6A.

### DETAILED DESCRIPTION OF THE EMBODIMENTS

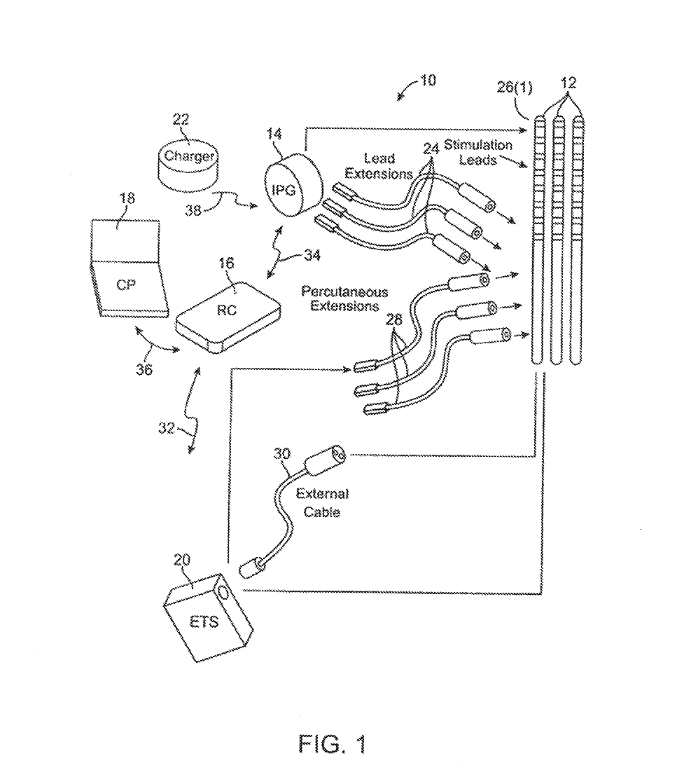

Referring to FIG. 1, an exemplary neuromodulation system 10 generally includes a plurality of modulation leads 12 (in this case, three), an implantable pulse generator (IPG) 14 (or alternatively RF receiver-stimulator), an external remote control (RC) 16, a Clinician's Programmer (CP) 18, an External Trial Stimulator (ETS) 20, and an external charger 22. As will be described in further detail below, the neuromodulation system 10 can be used to electrically modulate intraosseous nerve fibers, and in the exemplary case, basivertebral nerve fibers, for treating chronic back pain. While the description of systems and methods of stimulating intraosseous nerve fibers will be directed to intraosseous nerve fibers of the vertebrae, and in particular, the basivertebral nerve fibers located within the vertebrae, it is to be understood that the systems and methods of stimulating intraosseous nerve fibers of the disclosure may be used, or performed, in connection with any intraosseous nerve fibers, e.g., nerve fibers located within the pelvis, the femur, the fibula, the tibia, humerus, ulna, radius, or any other bone.

The IPG 14 is physically connected via one or more lead extensions 24 to the modulation leads 12, which carry multiple electrodes 26 arranged in an array. The modulation leads 12 are illustrated as percutaneous leads in FIG. 1, although a surgical paddle lead can also be used in place of the percutaneous leads. As will be described in further detail below, the IPG 14 includes pulse generation circuitry that delivers electrical modulation energy in the form of a pulsed electrical waveform (i.e., a temporal series of electrical pulses) to the electrodes 26 in accordance with at least a first set of modulation parameters.

The ETS 20 may also be physically connected via the percutaneous lead extensions 28 and external cable 30 to the neuromodulation leads 12. The ETS 20, which has similar pulse generation circuitry as the IPG 14, also delivers electrical modulation energy in the form of a pulse electrical waveform to the electrodes 26, based on a first set of modulation parameters. The IPG 14 may use the first set of parameters. Similarly, a second set of parameters may be used by the ETS 20, which may be same, or different, to that of the first set of parameters. The major difference between the ETS 20 and the IPG 14 is that the ETS 20 is a non-implantable device that is used on a trial basis after the neuromodulation leads 12 have been implanted, prior to implantation of the IPG 14, to test the responsiveness of the modulation that is to be provided. Thus, any functions described herein with respect to the IPG 14 can likewise be performed with respect to the ETS 20.

The RC 16 may be used to telemetrically control the ETS 20 via a bi-directional RF communications link 32. Once the IPG 14 and neuromodulation leads 12 are implanted, the RC 16 may be used to telemetrically control the IPG 14 via a bi-directional RF communications link 34. Such control allows the IPG 14 to be turned on or off and to be programmed with different modulation parameter sets. The IPG 14 may also be operated to modify the programmed modulation parameters to actively control the characteristics of the electrical modulation energy output by the IPG 14. As will be described in further detail below, the CP 18 includes a processor (not shown) and provides clinician detailed modulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed modulation parameters provided by the CP 18 are also used to program the RC 16, so that the modulation parameters can be subsequently modified by operation of the RC 16 in a stand-alone mode (i.e., without the assistance of the CP 18). The charger 22 may also communicate with the IPG 14 via a communications link 38.

For purposes of brevity, the details of the RC 16, CP 18, ETS 20, and external charger 22 will not be described herein. Details of exemplary embodiments of these devices are disclosed in U.S. Pat. No. 6,895,280, which is expressly incorporated herein by reference.

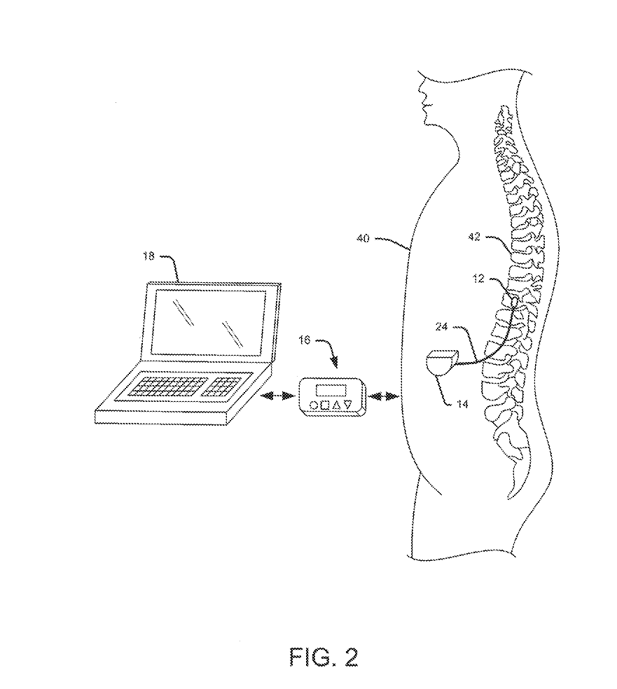

As shown in FIG. 2, the modulation leads 12 are implanted within the spinal column 42 of a patient 40. As will be described in further detail below, the preferred placement of the leads 12 is within one of the vertebral bodies 108 in the thoracic or lumbar region of the spinal column 42. Due to the lack of space near the location where the leads 12 exit the spinal column 42, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the leads 12. As there shown, the CP 18 communicates with the IPG 14 via the RC 16.

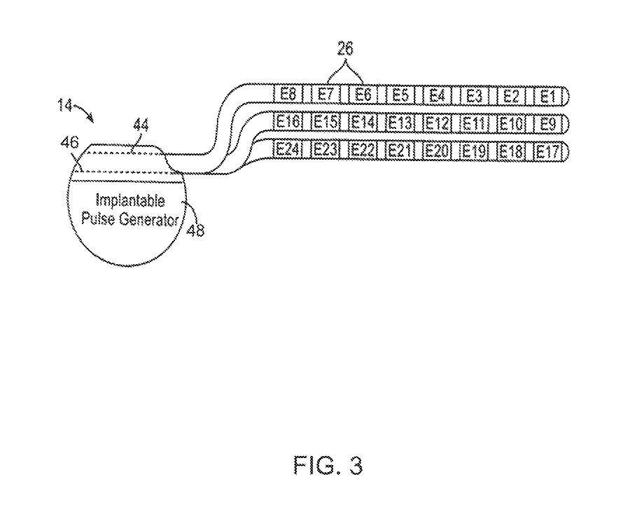

Referring now to FIG. 3, the external features of the modulation leads 12 and the IPG 14 will be briefly described. Each of the modulation leads 12 has eight electrodes 26 (respectively labeled E1-E8, E9-E16, and E17-E24). The actual number and shape of leads and electrodes will, of course, vary according to the intended application. Further details describing the construction and method of manufacturing percutaneous modulation leads are disclosed in U.S. patent application Ser. No. 11/689,918, entitled “Lead Assembly and Method of Making Same,” and U.S. patent application Ser. No. 11/565,547, entitled “Cylindrical Multi-Contact Electrode Lead for Neural Stimulation and Method of Making Same,” the disclosures of which are expressly incorporated herein by reference.

In the exemplary embodiments illustrated in FIG. 3, the IPG 14 includes an outer case 48 for housing the electronic and other components (described in further detail below). The outer case 48 is composed of an electrically conductive, biocompatible material, such as titanium, and forms a hermetically sealed compartment, wherein the internal electronics are protected from the body tissue and fluids. In some cases, the outer case 48 may serve as an electrode. The IPG 14 further comprises a connector 46 to which the proximal ends of the modulation leads 12 mate in a manner that electrically couples the electrodes 26 to the internal electronics (described in further detail below) within the outer case 48. To this end, the connector 46 includes one or more ports (three ports 44 or three percutaneous leads or one port for the surgical paddle lead) for receiving the proximal end(s) of the modulation lead(s) 12. In the case, where the lead extensions 24 are used, the port(s) 44 may instead receive the proximal ends of such lead extensions 24.

The IPG 14 includes pulse generation circuitry that provides electrical modulation energy in the form of a pulsed electrical waveform to the electrodes 26 in accordance with a set of modulation parameters programmed into the IPG 14. Such modulation parameters may include electrode combinations, which define the electrodes that are activated as anodes (positive), cathodes (negative), and turned off (zero), percentage of modulation energy assigned to each of the electrodes 26 (fractionalized electrode configurations). The modulation parameters may further include certain electrical pulse parameters, which define the pulse amplitude (measured in milliamps or volts depending on whether the IPG 14 supplies constant current or constant voltage to the electrodes 26), pulse width (measured in microseconds), pulse rate (measured in pulses per second), and burst rate (measured as the modulation on duration X and modulation off duration Y).

Electrical modulation will occur between two (or more) activated electrodes, one of which may be the IPG case 48. Modulation energy may be transmitted to the tissue in a monopolar or multipolar (e.g., bipolar, tripolar, etc.) fashion. Monopolar modulation occurs when a selected one of the lead electrodes 26 is activated along with the case 48 of the IPG 14, so that modulation energy is transmitted between the selected electrode 26 and the case 48. Bipolar modulation occurs when two of the lead electrodes 26 are activated as anode and cathode, so that modulation energy is transmitted between the selected electrodes 26. For example, an electrode on one lead 12 may be activated as an anode at the same time that an electrode on the same lead or another lead 12 is activated as a cathode. Tripolar modulation occurs when three of the lead electrodes 26 are activated, two as anodes and the remaining one as a cathode, or two as cathodes and the remaining one as an anode. For example, two electrodes on one lead 12 may be activated as anodes at the same time that an electrode on another lead 12 is activated as a cathode.

The modulation energy may be delivered between electrodes as monophasic electrical energy or multiphasic electrical energy. Monophasic electrical energy includes a series of pulses that are either all positive (anodic) or all negative (cathodic). Multiphasic electrical energy includes a series of pulses that alternate between positive and negative. For example, multiphasic electrical energy may include a series of biphasic pulses, with each biphasic pulse including a cathodic (negative) modulation pulse and an anodic (positive) recharge pulse that is generated after the modulation pulse to prevent direct current charge transfer through the tissue, thereby avoiding electrode degradation and cell trauma. That is, charge is conveyed through the electrode-tissue interface via current at an electrode during a modulation period (the length of the modulation pulse), and then pulled back off the electrode-tissue interface via an oppositely polarized current at the same electrode during a recharge period (the length of the recharge pulse).

As briefly discussed above, the modulation leads 12 may be implanted within one or more vertebral bodies 108 to allow modulation of the basivertebral nerve fibers for the purpose of treating back pain.

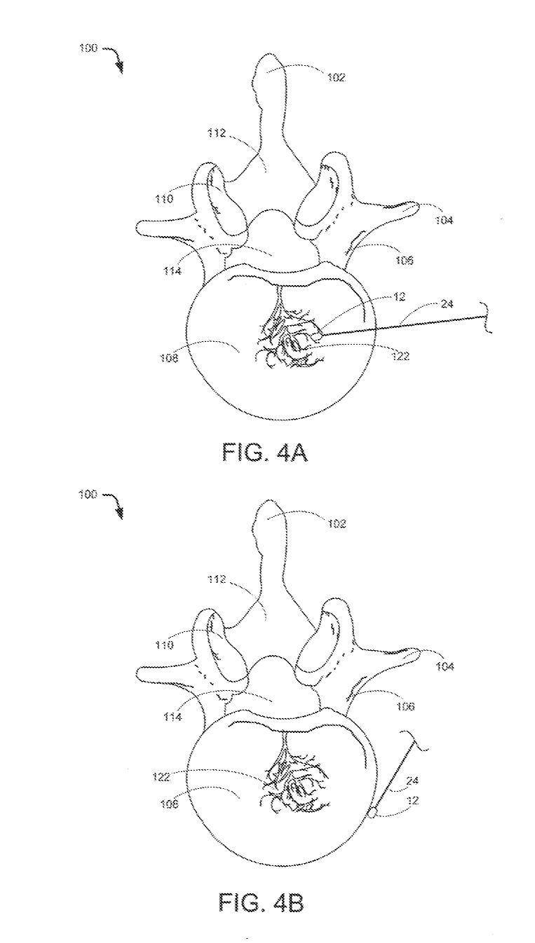

Referring now to FIGS. 4A-4B, one method of modulating the basivertebral nerve fibers 122 using the system 10 will now be described. As shown, the vertebrae 100 includes the vertebral body 108, the vertical arch (not shown) comprising the lamina 112 and the pedicle or root 106, the transverse process 104, the spinous process or spine 102, the inferior articular process 116, the superior articular process 110, the vertebral foramen 114, the superior vertebral notch 118, and the inferior vertebral notch 120. Basivertebral nerve fibers 122 are disposed within the vertebral body 108.

As shown in FIG. 4A, one of the modulation leads 12 may be implanted within the vertebral body 108, such that at least one of the electrodes 26 (as shown in FIG. 1) is located at a target site adjacent the basivertebral nerve fibers 122 that are transmitting the pain traffic to the brain. Additional modulation leads 12 may be implanted within the same vertebral body 108 or another vertebral body 108.

Alternatively, as shown in FIG. 4B, one of the modulation leads 12 may be implanted outside the vertebral body 108, such that at least one of the electrodes 26 (as shown in FIG. 1) is located at a target site on the external surface of the vertebral body 108. In this case, the modulation lead 12 may be a surgical paddle lead that conforms to the external surface of the vertebral body 108.

Once the modulation lead or leads 12 are implanted in the patient, such that one or more of the electrodes 26 are located at the target site or sites in or around the vertebral body or bodies 108, electrical modulation energy can be delivered from the IPG 14 to the modulation lead(s) 12 to electrically modulate the basivertebral nerve fibers 122, thereby treating the pain. In exemplary embodiments, the basivertebral nerve fibers 122 may be modulated using subthreshold, hyperpolarizing, anodic pre-pulsing (conditioning), continuous or burst modulation to hyperpolarize neurons closest to an active electrode. High frequency rates of 2-30 kHz may be used to block the pain traffic within the basivertebral nerve fibers 122. In an exemplary burst mode, rates above 100 Hz may be used to create activity dependent hyperpolarization and increase the relative threshold for activation. Exemplary pulses that may be used include charge-balanced sinusoidal, rectangular, triangular, exponential, trapezoidal, sawtooth, or spiked pulses, and may be either monophasic or biphasic. The pulse complexes may be symmetrical or asymmetrical. Programming strategies that focus the modulation field, such as narrow biopoles and tripoles, may be used such that non-targeted neural tissue is not inadvertently activated. Further, interlead bipole configurations can be used to maximize current flow in the entire vertebral body. The neuromodulation system 10 may be used on a temporary or permanent basis. The modulation leads 12 can be explanted and discarded right after use, or alternatively, the modulation leads 12 may be safely implanted for an extended duration prescribed by the treating practitioner.

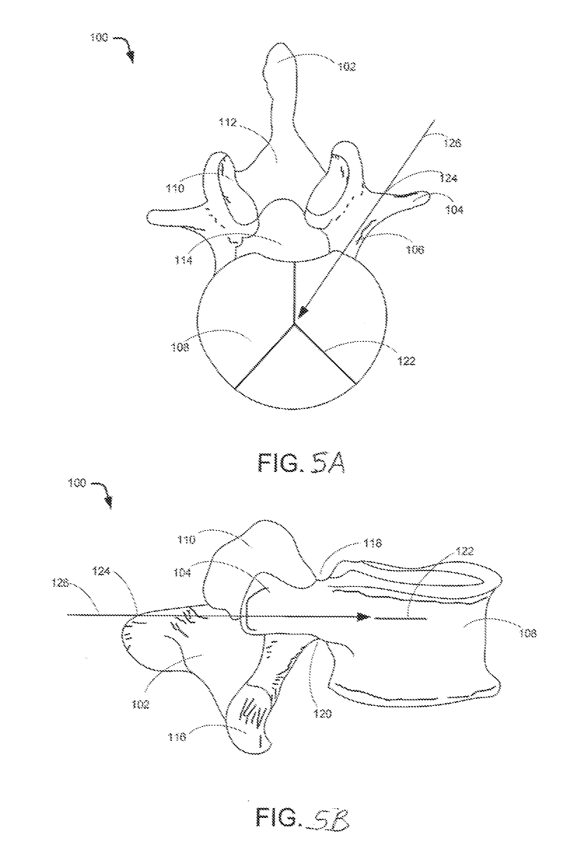

Referring now to FIGS. 5A-5B, a transpedicular approach may be employed to deliver one or more lead (s) 12 within a vertebral body 108. Such approach facilitates placement of the lead 12 adjacent an internal bone surface of the vertebral body 108 such that the lead 12 can stimulate the basivertebral nerves 122. To accomplish this, the lead 12 may enter the vertebral body 108 to a predetermined depth. Utilizing a conventional tool, such as a drill, a passageway may be created starting at the point of entry 124 in a direction of penetration (arrow 126). The passageway is created along arrow 126 through the transverse process 104, the pedicle 106, and ultimately, the vertebral body 108 until the passageway contacts, or is in close proximity to, the basivertebral nerve fibers 122 (located at the tip of arrow 126). Once the passageway is created, conventional tools, such as a cannula and/or stylet, can be used to guide leads 12 to contact, or otherwise be in close proximity to, the basivertebral nerve fibers 122.

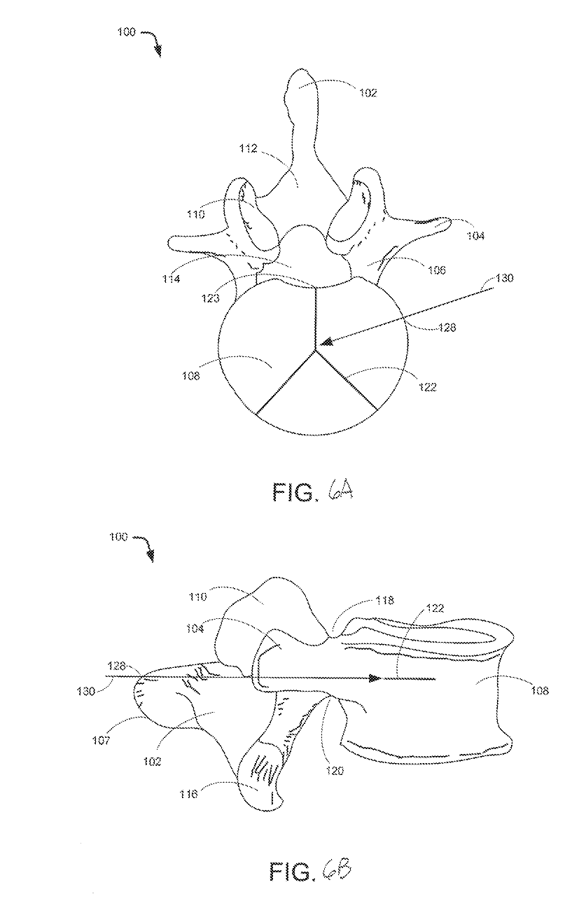

In an alternate embodiment, a posterolateral approach for penetrating the vertebral cortex to access the basivertebral nerve fibers 122 is employed, as shown in FIGS. 6A-6B. In this exemplary embodiment, a passageway (not shown) is created at the point of entry 128 in the direction of penetration, i.e., arrow 130. The passageway is created along arrow 130 through the posterior end 107 of the vertebral body 108 beneath the transverse process 104 until the passageway contacts, or is in close proximity to, the basivertebral nerve fibers 122 (located at the tip of arrow 130).

It is to be understood that the disclosure is not limited to the exact details of construction, operation, exact materials, or exemplary embodiments shown and described, as obvious modifications and equivalents will be apparent to one skilled in the art. For example, while 5A-5B and 6A-6B represent two preferred approaches, it will be appreciated by those of ordinary skill in the art that alternate approaches may be made depending upon the clinical setting. For example, the surgeon may elect not to cut or penetrate the vertebral bone but instead access, and stimulate, the basivertebral nerve fibers via, or adjacent, the vertebral foramen 114 at, or in close proximity to, the exit point of the basivertebral nerve fibers from the bone.

Although particular embodiments of the present disclosure have been shown and described, it will be understood that it is not intended to limit the present disclosure to the preferred embodiments, and it will be obvious to those skilled in the art that various changes and modifications may be made without departing from the spirit and scope of the present disclosure. Thus, the present disclosure are intended to cover alternatives, modifications, and equivalents, which may be included within the spirit and scope of the present disclosure as defined by the claim

What is claimed is:

1. A method for treating a patient having pain, the method comprising:
applying electrical modulation energy to a target site adjacent an intraosseous nerve fiber of the patient to modulate pain traffic within the intraosseous nerve fiber, thereby treating the pain.

2. The method of claim 1, wherein the pain traffic is nociceptive pain traffic.

3. The method of claim 1, wherein the intraosseous nerve fiber is a basivertebral nerve fiber, and the pain is back pain.

4. The method of claim 1, wherein the target site is in a vertebral body of the patient.

5. The method of claim 1, wherein the target site is in one of a pelvis, femur, fibula, tibia, humerus, ulna, and radius of the patient.

6. The method of claim 1, wherein the target site is within a bone in which the intraosseous nerve fiber innervates.

7. The method of claim 1, wherein the target site is on an external surface of a bone in which the intraosseous nerve fiber innervates.

8. The method of claim 1, wherein the application of the electrical modulation energy to the target site reduces or prevents the pain traffic within the intraosseous nerve fiber.
